(19) United States
(12) Patent Application Publication
(10) Pub. No.: US 2020/0085885 A1
(43) Pub. Date: Mar. 19, 2020

(54) PROBIOTIC COMPOSITIONS AND METHODS

(71) Applicant: CORNELL UNIVERSITY, Ithaca, NY (US)

(72) Inventor: Rodrigo Carvalho BICALHO, Ithaca, NY (US)

(21) Appl. No.: 16/471,860

(22) PCT Filed: Dec. 18, 2017
(86) PCT No.: PCT/US2017/067035

§ 371 Date: Jun. 20, 2019

Related U.S. Application Data
(60) Provisional application No. 62/437,406, filed on Dec. 21, 2016.

(51) Int. Cl. A61K 35/741; A23K 10/18; A23K 50/10; A23K 50/60; A61K 35/747; A61K 35/742; A61K 35/744; A61K 35/745

(52) U.S. Cl. CPC A61K 35/741; A23K 10/18; A23K 50/10; A61K 35/745; A61K 35/747; A61K 35/742; A61K 35/744; A23K 50/60

(57) ABSTRACT

The present invention relates to probiotic compositions and methods of using such compositions. In particular, the present invention provides methods of using Faecalibacterium spp. to increase milk production in animals.

### CROSS REFERENCE TO RELATED APPLICATION

This application claims priority to and the benefit of U.S. Provisional Application No. 62/437,406, filed Dec. 21, 2016, which is hereby incorporated by reference in its entirety.

### FIELD OF INVENTION

The present invention relates to probiotic compositions and methods of using such compositions. In particular, the present invention provides methods of using Faecalibacterium spp. to increase milk production in animals.

### BACKGROUND OF THE INVENTION

Gut microbiota is known to have a role in shaping key aspects of postnatal life, such as the development of the immune system (Mazmanian et al., (2005) Cell 122(1): 107-118; Peterson et al., (2007) Cell Host Microbe 2(5): 328-339), and influencing the host's physiology, including energy balance. Transplanting the gut microbiota from normal mice into germ-free recipients increased their body fat without any increase in food consumption, raising the possibility that the composition of the microbial community in the gut affects the amount of energy extracted from the diet (Backhed et al., (2004) Proc Natl Acad Sci USA 101(44): 15718-15723). There is at least one type of obesity-associated gut microbiome characterised by higher relative abundance of Firmicutes or a higher Firmicutes to Bacteroidetes ratio (Ley et al., (2005) Proc Natl Acad Sci USA 102(31): 11070-11075; Tumbaugh et al., (2006) Nature 444(7122): 1027-1031). The role of intestinal microbiota in disease has also been shown. Gut microbes serve their host by functioning as a key interface with the environment; for example, they can protect the host organism from pathogens that cause infectious diarrhea. A decreased diversity of fecal microbiota and specifically a reduced diversity of Firmicutes in Crohn's disease patients has been reported (Manichanh et al., (2006) Gut 55(2): 205-211), while it was recently shown that Faecalibacterium prausnitzii displays anti-inflammatory action and can potentially be used for the treatment of this disease (Sokol et al., (2008) Proc Natl Acad Sci USA 105(43): 16731-16736).

Efficient growth of pre-weaned dairy calves together with low incidence of disease (especially diarrhea and pneumonia) are prerequisites for their optimal performance after weaning and contribute in the profitability of a dairy enterprise. For every 1 kg of pre-weaning average daily gain, milk yield increased by 1,113 kg in the first lactation (Soberon et al., (2012) J Dairy Sci 95(2): 783-793). The notion that calves' intestinal microbiota profiles are probably related with growth and disease already exists. Probiotics, bacteria with a beneficial effect on animals' intestinal health, have been found to have antidiarrheal capacities and enhance growth rates in calves (Donovan et al., (2002) J Dairy Sci 85(4): 947-950; Timmerman et al., (2005) J Dairy Sci 88(6): 2154-2165).

However, methods for improving milk production of animals are still needed.

### SUMMARY OF THE INVENTION

The present invention relates to probiotic compositions and methods of using such compositions. In particular, the present invention provides methods of using Faecalibacterium spp. to increase milk production in animals.

For example, in some embodiments, the present invention provides a method of improving milk production or future milk production in an animal comprising administering to the animal a composition comprising one or more Faecalibacterium spp. (e.g., including but not limited to, Faecalibacterium prausnitzii). In some embodiments, the composition comprises one or more Faecalibacterium spp. in an amount effective to increase milk production in the animal. The present invention is not limited to a particular animal. Examples include, but are not limited to, domestic animals (e.g., cattle (e.g., calf), sheep, swine, or horses). In some embodiments, the animal is less than 1 week, one month, or two months of age. In some embodiments, the composition is formulated as a powder, bolus, gel, drench, or capsule. In some embodiments, the composition is provided as part of a milk replacer. In some embodiments, the composition is coadministered with at least a second probiotic organism (e.g., including but not limited to, Lactobacillus acidophilus, L. lactis, L. plantarum, L. casei, Bacillus subtilis, B. lichenformis, Enterococcus faecium, Bifidobacterium bifidum, B. longum, B. thermophilum, Propionibacterium jensenii, yeast, or combinations thereof). In some embodiments, the composition is formulated with an additional additive (e.g., including but not limited to, an energy substrate, a mineral, a vitamin, or combinations thereof).

Additional embodiments provide a probiotic composition comprising Faecalibacterium spp. in combination with a milk protein. In some embodiments, the composition is a powder or a milk replacer. In some embodiments, the composition further comprises an energy substrate, a mineral, a vitamin, or at least a second probiotic organism (e.g., including but not limited to, Lactobacillus acidophilus, L. lactis, L. plantarum, L. casei, Bacillus subtilis, B. lichenformis, Enterococcus faecium, Bifidobacterium bifidum, B. longum, B. thermophilum, Propionibacterium jensenii, and yeast, or combinations thereof).

The present invention further provides a probiotic composition for administration to a domestic animal comprising Faecalibacterium spp. in combination with an additional additive selected from, for example, an energy substrate, a mineral, a vitamin, at least a second probiotic organism (e.g., including but not limited to, Lactobacillus acidophilus, L. lactis, L. plantarum, L. casei, Bacillus subtilis, B. lichenformis, Enterococcus faecium, Bifidobacterium bifidum, B. longum, B. thermophilum, Propionibacterium jensenii, and yeast, or combinations thereof). In some embodiments, the composition is formulated as an oral delivery vehicle powder, bolus, gel, drench, or capsule, suitable for administration to a domestic animal. In some embodiments, the composition is provided in an amount effective to improve milk production or future milk production in an animal.

The present invention also provides the use of any of the aforementioned compositions to improve milk production or future milk production in an animal.

Further embodiments of the present invention provide a method of supplementing the diet of a domestic animal comprising adding Faecalibacterium spp. to the diet of the domestic animal.

Additional embodiments are described herein.

### DESCRIPTION OF THE FIGURES

FIG. 1: Faecalibacterium mean relative abundance. Field trial. Faecalibacterium mean relative abundance (Y axis, %) for each treatment group (control and FPTRT) over their 1^{st}, 3^{rd}, 5^{th} and 7^{th} week of life(X axis). The error bars represent the standard errors of the means.

FIG. 2: Effect of Faecalibacterium prausnitzii versus negative control treatments of neonatal Holstein heifer calves on future milk production during the first 5 weeks of the first lactation. Calves treated with Faecalibacterium prausnitzii produced significantly more milk when compared with negative controls (P-value<0.05).

### DEFINITIONS

To facilitate an understanding of the present invention, a number of terms and phrases are defined below:

As used herein, the term “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

As used herein, the term “prokaryotes” refers to a group of organisms that usually lack a cell nucleus or any other membrane-bound organelles. In some embodiments, prokaryotes are bacteria. The term “prokaryote” includes both archaea and eubacteria.

As used herein, the term “in vitro” refers to an artificial environment and to processes or reactions that occur within an artificial environment. In vitro environments can consist of, but are not limited to, test tubes, microtiter plates, and the like. The term “in vivo” refers to the natural environment (e.g., an animal or a cell) and to processes or reactions that occur within a natural environment.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

Mammals are defined herein as all animals (e.g., human or non-human animals) that have mammary glands and produce milk.

As used herein, a “dairy animal” refers to a milk producing non-human mammal that is larger than a laboratory rodent (e.g., a mouse). In preferred embodiments, the dairy animals produce large volumes of milk and have long lactating periods (e.g., cows or goats).

A “subject” is an animal such as vertebrate, preferably a domestic animal or a mammal. Mammals are understood to include, but are not limited to, murines, simians, humans, bovines, cervids, equines, porcines, canines, felines etc.

An “effective amount” is an amount sufficient to effect beneficial or desired results. An effective amount can be administered in one or more administrations,

“Co-administration” refers to administration of more than one agent or therapy to a subject. Co-administration may be concurrent or, alternatively, the chemical compounds described herein may be administered in advance of or following the administration of the other agent(s). One skilled in the art can readily determine the appropriate dosage for co-administration. When co-administered with another therapeutic agent, both the agents may be used at lower dosages. Thus, co-administration is especially desirable where the claimed compounds are used to lower the requisite dosage of known toxic agents.

As used herein, the term “toxic” refers to any detrimental or harmful effects on a cell or tissue.

A “pharmaceutical composition” is intended to include the combination of an active agent with a carrier, inert or active, making the composition suitable for diagnostic or therapeutic use in vivo, in vivo or ex vivo.

As used herein, the term “pharmaceutically acceptable carrier” encompasses any of the standard pharmaceutical carriers, such as a phosphate buffered saline solution, water, and an emulsion, such as an oil/water or water/oil emulsion, and various types of wetting agents. The compositions also can include stabilizers and preservatives. For examples of carriers, stabilizers and adjuvants see Martin, Remington's Pharmaceutical Sciences, 15th Ed., Mack Publ. Co., Easton, Pa. (1975).

“Pharmaceutically acceptable salt” as used herein, relates to any pharmaceutically acceptable salt (acid or base) of a compound of the present invention, which, upon administration to a recipient, is capable of providing a compound of this invention or an active metabolite or residue thereof. As is known to those of skill in the art, “salts” of the compounds of the present invention may be derived from inorganic or organic acids and bases. Examples of acids include hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic and benzenesulfonic acid. Other acids, such as oxalic, while not in themselves pharmaceutically acceptable, may be employed in the preparation of salts useful as intermediates in obtaining the compounds of the invention and their pharmaceutically acceptable acid.

As used herein, the term “nutraceutical,” refers to a food substance or part of a food, which includes a probiotic bacterium. Nutraceuticals can provide medical or health benefits, including the prevention, treatment, or cure of a disorder.

The terms “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. Also included within this term are prokaryotic organisms that are gram negative or gram positive. “Gram negative” and “gram positive” refer to staining patterns with the Gram-staining process that is well known in the art. (See e.g., Finegold and Martin, Diagnostic Microbiology, 6th Ed., CV Mosby St. Louis, pp. 13-15 [1982]). “Gram positive bacteria” are bacteria that retain the primary dye used in the Gram stain, causing the stained cells to appear dark blue to purple under the microscope. “Gram negative bacteria” do not retain the primary dye used in the Gram stain, but are stained by the counterstain. Thus, gram negative bacteria appear red.

### DETAILED DESCRIPTION OF THE INVENTION

The present invention relates to probiotic compositions and methods of using such compositions. In particular, the present invention provides methods of using Faecalibacterium spp. to increase milk production in animals.

Faecalibacterium prausnitzii belongs to the phylum Firmicutes and is an obligate anaerobic, Gram-positive, rod-shaped, butyrate producing microorganism [5,6] that is abundant in the feces of several animal species [7-13]. In humans, high levels of F. prausnitzii were associated with obesity [14], while a low abundance of F. prausnitzii was linked to Inflammatory Bowel Disease (IBD, Crohn's disease [15,16] and ulcerative colitis [17]). F. prausnitzii has anti-inflammatory properties, which have been demonstrated in vitro with cultured cells and in vivo with trinitrobenzenesulfonic acid (TNBS)-induced colitis in mice models [16,18-20]. F. prausnitzii induces the production of the anti-inflammatory cytokine IL-10 and reduces the secretion of the pro-inflammatory cytokines IFN-γ and IL-12 [20]. Furthermore, F. prausnitzii and its supernatant decreased the severity of colitis in IBD mice models [16,18]. Additionally, the butyrate produced by F. prausnitzii is both an energy source to enterocytes and act as an anti-inflammatory agent [21].

In preweaned Holstein calves, higher relative abundance of F. prausnitzii in the first week of life was associated with enhanced weight gain and reduced incidence of diarrhea [10]. A recent study isolated 203 F. prausnitzii isolates from the feces of calves and piglets [5]. In that study, 40 genetically distinct F. prausnitzii isolates were selected for further characterization. A large variability was observed among isolates for in vitro short chain fatty acids (SCFA) metabolism, growth, antibiotic resistance, and sensitivity to low pH and bile salts. Based on this data, 4 isolates with desirable characteristics were selected and used as part of a probiotic cocktail in the in vivo studies described herein.

Experiments described herein demonstrated the effects of the oral administration of F. prausnitzii to neonatal Holstein calves on future milk production.

Accordingly, embodiments of the present invention provide probiotic compositions comprising Faecalibacterium species and uses of such compositions in increasing milk production or future milk production in animals.

### I. Compositions and Kits

In some embodiments, the present invention provides probiotic compositions and kits. In some embodiments, probiotic compositions comprise one or more Faecalibacterium spp. The present invention is not limited to a particular one or more Faecalibacterium spp. Examples include, but are not limited to, Faecalibacterium prausnitzii.

In some embodiments, compositions comprise one or more (e.g., 2 or more, 5 or more, 10 or more, etc.) additional strains of bacteria or other microorganisms (e.g., probiotic microorganisms). Examples include, but are not limited to, Lactobacillus acidophilus, L. lactis, L. plantarum, L. casei, Bacillus subtilis, B. lichenformis, Enterococcus faecium, Bifidobacterium bifidum, B. longum, B. thermophilum, Propionibacterium jensenii, yeast, or combinations thereof. In some embodiments, multiple strains of the same bacteria are utilized in combination.

In some embodiments, compositions comprise one or more additional components (e.g., including but not limited to, additional additive selected from the group consisting of an energy substrate, a mineral, a vitamin, or combinations thereof).

In some embodiments, bacteria are live cells or freeze-dried cells. Freeze-dried bacteria can be stored for several years with maintained viability. In certain applications, freeze-dried bacteria are sensitive to humidity. One way of protecting the bacterial cells is to store them in oil. The freeze dried bacterial cells can be mixed directly with a suitable oil, or alternately the bacterial cell solution can be mixed with an oil and freeze dried together, leaving the bacterial cells completely immersed in oil. Suitable oils may be edible oils such as olive oil, rapeseed oil which is prepared conventionally or cold-pressed, sunflower oil, soy oil, maize oil, cotton-seed oil, peanut oil, sesame oil, cereal germ oil such as wheat germ oil, grape kernel oil, palm oil and palm kernel oil, linseed oil. The viability of freeze-dried bacteria in oil is maintained for at least nine months. Optionally live cells can be added to one of the above oils and stored.

In some embodiments, the compositions are part of a milk replacer (e.g., for administration to a neonatal or young animal). In some embodiments, compositions comprise one or more probiotic bacteria as described herein in combination with a milk protein (e.g., caseins or whey proteins).

In some embodiments, compositions are added to nutraceuticals, food products, or foods. In some embodiments, to give the composition or nutraceutical a pleasant taste, flavoring substances such as for example mints, fruit juices, licorice, Stevia rebaudiana, steviosides or other calorie free sweeteners, rebaudioside A, essential oils like eucalyptus oil, or menthol can optionally be included in compositions of embodiments of the present invention.

In some compositions embodiments, compositions are formulated in pharmaceutical compositions. The bacteria of embodiments of the invention may be administered alone or in combination with pharmaceutically acceptable carriers or diluents, and such administration may be carried out in single or multiple doses.

Compositions may, for example, be in the form of tablets, resolvable tablets, capsules, bolus, drench, pills sachets, vials, hard or soft capsules, aqueous or oily suspensions, aqueous or oily solutions, emulsions, powders, granules, syrups, elixirs, lozenges, reconstitutable powders, liquid preparations, creams, troches, hard candies, sprays, chewing-gums, creams, salves, jellies, gels, pastes, toothpastes, rinses, dental floss and tooth-picks, liquid aerosols, dry powder formulations, HFA aerosols or organic or inorganic acid addition salts.

The pharmaceutical compositions of embodiments of the invention may be in a form suitable for oral, topical, buccal administration. Depending upon the disorder and subject to be treated and the route of administration, the compositions may be administered at varying doses.

For oral or buccal administration, bacteria of embodiments of the present invention may be combined with various excipients. Solid pharmaceutical preparations for oral administration often include binding agents (for example syrups, acacia, gelatin, tragacanth, polyvinylpyrrolidone, sodium lauryl sulphate, pregelatinized maize starch, hydroxypropyl methylcellulose, starches, modified starches, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone and sodium alginate), disintegrants (such as starch and preferably corn, potato or tapioca starch, alginic acid and certain complex silicates, polyvinylpyrrolidone, gelatin, acacia, sodium starch glycollate, microcrystalline cellulose, crosscarmellose sodium, crospovidone, hydroxypropyl methylcellulose and hydroxypropyl cellulose), lubricating agents (such as magnesium stearate, sodium lauryl sulfate, talc, silica polyethylene glycol waxes, stearic acid, palmitic acid, calcium stearate, carnuba wax, hydrogenated vegetable oils, mineral oils, polyethylene glycols and sodium stearyl fumarate) and fillers (including high molecular weight polyethylene glycols, lactose, calcium phosphate, glycine magnesium stearate, starch, rice flour, chalk, gelatin, microcrystalline cellulose, calcium sulphate, and lactitol). Such preparations may also include preservative agents and anti-oxidants.

Liquid compositions for oral administration may be in the form of, for example, emulsions, syrups, or elixirs, or may be presented as a dry product for reconstitution with water or other suitable vehicle before use. Such liquid compositions may contain conventional additives such as suspending agents (e.g. syrup, methyl cellulose, hydrogenated edible fats, gelatin, hydroxyalkylcelluloses, carboxymethylcellulose, aluminium stearate gel, hydrogenated edible fats) emulsifying agents (e.g. lecithin, sorbitan monooleate, or acacia), aqueous or non-aqueous vehicles (including edible oils, e.g. almond oil, fractionated coconut oil) oily esters (for example esters of glycerine, propylene glycol, polyethylene glycol or ethyl alcohol), glycerine, water or normal saline; preservatives (e.g. methyl or propyl p-hydroxybenzoate or sorbic acid) and conventional flavouring, preservative, sweetening or colouring agents. Diluents such as water, ethanol, propylene glycol, glycerin and combinations thereof may also be included.

Other suitable fillers, binders, disintegrants, lubricants and additional excipients are well known to a person skilled in the art.

In some embodiments, bacteria are spray-dried. In other embodiments, bacteria are suspended in an oil phase and are encased by at least one protective layer, which is water-soluble (water-soluble derivatives of cellulose or starch, gums or pectins; See e.g., EP 0 180 743, herein incorporated by reference in its entirety).

In some embodiments, the present invention provides kits, pharmaceutical compositions, or other delivery systems for use in increasing milk production or future milk productionin an animal. The kit may include any and all components necessary, useful or sufficient for research or therapeutic uses including, but not limited to, one or more probiotic bacteria, pharmaceutical carriers, and additional components useful, necessary or sufficient for increasing milk production or future milk production in an animal. In some embodiments, the kits provide a sub-set of the required components, wherein it is expected that the user will supply the remaining components. In some embodiments, the kits comprise two or more separate containers wherein each container houses a subset of the components to be delivered.

Optionally, compositions and kits comprise other active components in order to achieve desired therapeutic effects.

### II. Therapeutic and Supplement Uses

Embodiments of the present invention provide compositions comprising probiotic bacteria (e.g., Faecalibacterium spp. alone or in combination with additional probiotic bacteria) (e.g., pharmaceutical, nutraceutical, or food compositions) for use in improving milk production or future milk production in an animal. In some embodiments, the animal is a domestic or agricultural animal (e.g., cow, sheep, goat, pig, etc.). In some embodiments, the animal is neonatal, newborn, or young. For example, in some embodiments, the animal is one day, 2, days, 3, days, 4 days, 5 days, 6 days, 1 week, 2 weeks, 3 weeks, one month, or 2 months of age, or from 1 day to one month old, 1 day to two months old, 1 day to 3 months old, 1 day to 4 months old, 1 day to 5 months old, one day to six months old or 1 day to 1 year old, or less than 1 week, 2 week, 3 weeks 1 months, 2 months, 3 months, 4 months, 5 months, 6 months or 12 months (what about at birth? why limit to 12 months only? What about maximum age at which cows produce milk?) old, although other ages and ranges falling within these guidelines are specifically contemplated.

In some embodiments, compositions comprising probiotic bacteria are administered once to an animal in need thereof. In other embodiments, compositions are administered on an ongoing, recurrent, or repeat basis (e.g., multiple times a day, once a day, once every 2, 3, 4, 5, or 6 days, once a week, etc.) for a period of time (e.g., multiple days, months, or weeks). Suitable dosages and dosing schedules are determined by one of skill in the art using suitable methods (e.g., those described in the experimental section below or known to one of skill in the art).

In some embodiments, the administration of compositions to a neonatal, newborn, or young animal increases future milk production (e.g., once the animal has reached sexual maturity).

### Experimental

The following examples are provided in order to demonstrate and further illustrate certain preferred embodiments and aspects of the present invention and are not to be construed as limiting the scope thereof.

### EXAMPLE 1

### Use of Faecalibacterium Prausnitzii in Holstein Calves Increases Future Milk Production

Materials and Methods

### Ethics Statement

This study was carried out in strict accordance with the recommendations of The Animal Welfare Act of 1966 (AWA) (P.L. 89-544) and its amendments 1970 (P.L. 91-579); 1976 (P.L. 94-279), and 1985 (P.L. 99-198) which regulate transport, purchase, care, and treatment of animals used in research. The research protocol was reviewed and approved by the Institutional Animal Care and Use Committee of Cornell University (Protocol number: 2012-0055). The administration of F. prausnitzii culture to calves housed on the commercial dairy farm was authorized by the farm owner, who was aware of all experimental procedures.

### Treatment Preparation

Four F. prausnitzii isolates were selected from a culture collection based on greater capacity for in vitro butyrate production, growth and tolerance to low pH and bile salts as previously evaluated by our research group [5]. The four isolates (ref numbers 34, 35, 1S, and 2S; Foditsch et al. (2014)) were cultured individually in a medium supplemented with 30% ruminal fluid as previously described [5]. The average colony forming units (CFU) of each isolate was 1.43×10⁷ CFU/mL. Equal volumes of the four cultures were mixed, frozen in 50 mL sterile disposable centrifuge tubes with 15% glycerol, and stored at −80° C. For quality assurance purposes, the CFU/mL was calculated at the time of administration; the average CFU was 1.34×10⁷ CFU/mL, confirming that a live bacteria culture was administered to the calves. The placebo given to control calves in the safety trial contained the same growth medium without the bacterial culture.

### Safety Trial

### Animals and Facilities

Thirty bull calves were obtained from a commercial dairy farm that milked 2,800 Holstein cows near Ithaca, N.Y., USA. Immediately after birth, calves were removed from the maternity pens and were placed in dry sawdust bedded pens. Four liters of pooled, non-pasteurized colostrum from primiparous cows was administered to calves by esophageal feeder. The primary researcher, a veterinarian, used an authorized van with individual transportation cages to transport the newborn calves to the College of Veterinary Medicine facility, where they were housed individually in 2.2×1.5 meters concrete stalls bedded with pine shavings. Calves were kept in the same stall during the 14 days of the research trial. Non-pasteurized whole milk was fed twice daily at approximately 10% of the body weight and water was available ad libitum. Stalls were kept clean and environmental enrichment utensils were used to minimize animal stress. No animal suffering was anticipated as a result of the trial, therefore analgesics and anesthetics were not administered. All animals were sold alive after the trial.

### Study Design and Data Collection

A randomized clinical trial design was used. Thirty calves were randomly allocated into one of four treatment groups as follows: oral control (n=5) calves received 80 mL of a placebo solution orally; oral treatment (n=10) calves received 80 mL of live culture of F. prausnitzii orally; rectal control (n=5) calves received 80 mL a placebo solution rectally; and rectal treatment (n=10) calves received 80 mL of live culture of F. prausnitzii rectally. Control groups received a placebo containing the growth medium without the bacterial culture. Oral treatments were administered through an esophageal tube and rectal treatments were given with a 6 cm drench tube attached to a syringe. Treatments were administered on the second day of life in order to avoid interactions between colostrum's immune cells and the bacteria administered. Due to the F. prausnitzii sensitivity to low pH [5], the treatments were administered 1 hour after milk feeding, when the abomasal pH increases approximately from 2 to 6 [22]. Calf health was assessed twice daily by the primary researcher for the following parameters; fecal consistency (0=well-formed; 1=semi-formed; 2=loose or watery feces not containing blood; and 3=loose or watery feces containing blood), dehydration (0=euhydrated; 1=skin tented 2 to 6s; 2=skin tented 6 to 10s; and 3=skin tented≥10s), attitude (0=alert; 1=depressed; and 2=non responsive) and appetite (0=normal; 1=consumed ½ bottle; 2=consumed ¼ bottle; and 3=forced fed). The effect of treatment on fecal consistency, dehydration, attitude and appetite scores was assessed using ordinal logistic regression models fitted in JMP Pro 11 (SAS Institute Inc., NC, USA). The independent variables offered to the model were treatment group, age in days, and interaction terms between treatment and age.

### Randomized Field Trial

### Farm and Management

Immediately after birth, female calves were removed from the maternity pens, weighed, and placed in dry sawdust bedded pens. Four liters of pooled non-pasteurized colostrum from primiparous cows was administered to calves by esophageal tubing and calves had their umbilicus dipped in 7% iodine solution.

Newborn calves were transported twice daily from the maternity area to the calf barn. Calves were housed in a green-house barn divided into 30 identical pens with positive ventilation. Pens were separated by steel gates and calves were moved by birth order into each pen until maximum capacity was reached (20 calves/pen). Calves remained in the same pen until weaning.

Calves were fed ad libitum acidified non-saleable milk using a fully automated system with 6 nipples per pen. Acidification was performed in a sealed stainless-steel tank where cold milk (5° C.) was mixed with organic acid under constant homogenization until a pH of 4.5 was reached. Acidified milk was directed to a smaller stainless-steel tank, warmed, and distributed to the pens. Acidified milk was offered to the calves from day one to 56 days of life. All calves were weaned by reducing the daily milk availability starting on day 42 until complete absence of acidified milk at 57 days of life. Water and solid feed (calf starter mix) were offered ad libitum to all calves.

Health-related events (e.g. otitis, pneumonia and severe diarrhea) were recorded and treated as needed by farm employees. One dose of the macrolide antibiotic Zuprevo (Merck Animal Health, Summit, N.J.) was given by the farm to all female calves at eight to 14 days of age as a metaphylactic for bovine respiratory disease. All calves were disbudded by heat cauterization at approximately four weeks of age.

### Study Design and Data Collection

The treatment administered was a live microorganism and cross-contamination between calves in the same group was possible. Therefore, all calves in the same pen were assigned to the same treatment group (oral treatment with F. prausnitzii (FPTRT) or control, at 5±2 days of life). The first group was randomly selected, and the subsequent groups were alternated between control and FPTRT, resulting in the same number of calves for each treatment group per week.

The rumen microbiota gradually changes from aerobic to anaerobic during the calves' first weeks of life [23-25], therefore we chose to treat calves in the field trial with two 40 ml doses of F. prausnitzii culture, one dose at treatment assignment (1^{st} week of life) and a second dose one week later, instead of only administering one 80 ml dose on the second day of life, to increase the chances of its colonization in the large intestine. The control calves did not receive a placebo treatment or sodium bicarbonate.

In a group feeding system it was not possible to determine the time each calf was fed and to account for the increase of the abomasal pH, as in the safety trial. Additionally, the milk fed in the commercial farm was acidified and, as mentioned previously, F. prausnitzii is highly sensitive to low pHs. Therefore, 130 mL of sodium bicarbonate (90 mg/mL) was administered orally to FPTRT calves to buffer the low pH of the abomasum before administering the culture. Sodium bicarbonate at 0.6% was used previously to increase the pH of fermented waste milk to 6.0 in a study evaluating feeding value of fermented waste milk [26]. In that study, calves received one of the four milk treatments (fresh milk, fresh waste milk, fermented waste milk or fermented waste milk with sodium bicarbonate) for 42 days and weight gain was not significantly different between groups. It was estimated that the dose of sodium bicarbonate, considering the milk present in the abomasum, would not have any affect, other than the neutralization of the abomasal pH prior to F. prausnitzii administration.

A total of 554 Holstein heifers were enrolled in the field trial, with 296 allocated to the control group and 258 to the FPTRT group. A subset of 35 calves/treatment was selected randomly for collection of fecal DNA through rectal swabs and evaluation of fecal microbiome. From these 70 calves, 45 calves (n=22, control; n=23, FPTRT) were selected randomly for evaluation of serum β-hydroxybutyrate (BHBA) concentrations. Blood samples were collected from the jugular vein and fecal samples were collected using rectal swabs on the 1^{st} (enrollment), 3^{rd}, 5^{th} and 7^{th} weeks of life. Blood samples were centrifuged at 3000× g for 10 minutes, after which serum was obtained. Serum and swabs were stored at −20° C. until assayed. Fecal consistency scores were recorded weekly using a four level scoring system, as described in the safety trial. Calves were weighed using a Waypig 15 digital scale (Vittetoe Inc., Keota, Iowa, USA) at birth and again at weaning (56±3 days of life; n=141 for the control group and n=146 for FPTRT group). Weight gain was calculated by subtracting the birth weight from the weight at weaning. The weight gain was divided by the age in days at the second weight (56±3 days) to obtain the average daily gain (ADG). Due to equipment constraints, weights of a subset of calves (303) were obtained. Severe diarrhea and death events records were acquired from the farm's software (Dairy-Comp 305; Valley Ag Software, Tulare, Calif., USA). Severe diarrhea was defined as dehydrated calves with loose or watery feces that were treated by the farm employees with oral electrolytes or intravenous fluids. Farm employees were blind to the treatment groups.

### DNA Extraction, Amplification and Purification

DNA of the fecal material from the four time points (1^{st}, 3^{rd}, 5^{th} and 7^{th} week of life) was extracted following the protocol previously used by Oikonomou et al. (2013). Briefly, each rectal swab was placed in 1.5 ml of nuclease-free water (Life Technologies, Grand Island, N.Y.) and vortexed for at least two minutes. The swab was then removed and the sample centrifuged for 10 min at 13,200× g. The supernatant was discarded and the remaining pellet was resuspended in 400 μl of nuclease-free water. Isolation of microbial genomic DNA was performed by using a QIAamp DNA minikit (Qiagen, Germantown, Md.) according to the manufacturer's instructions. Besides the proteinase K and the Buffer AL, 40 μl (10 mg/ml) of lysozyme (Sigma-Aldrich, St. Louis, Mo.) were added to the sample and the incubation at 56° C. was extended for 12 h. The DNA concentration and purity were evaluated by optical density using a NanoDrop ND-1000 spectrophotometer (NanoDrop Technologies, Rockland, Del., USA) at wavelengths of 230, 260 and 280 nm.

The 16S rRNA gene was amplified by PCR from individual metagenomic DNA samples using barcoded primers. For amplification of the V4 hypervariable region of the bacterial/archaeal 16S rRNA gene, primers 515F and 806R were used according to a previously described method optimized for the Illumina MiSeq platform (Illumina, Inc., San Diego, Calif., USA) [27]. The earth microbiome project [28] was used to select 280 different 12-bp error-correcting Golay barcodes for the 16S rRNA PCR, as previously described [27]. The 5′-barcoded amplicons were generated in triplicate using 14 DNA template, 2 X EconoTaq® Plus Green Master Mix (Lucigen®, Middleton, Wis., USA), and 5 μM of each primer. The PCR conditions for the 16S rRNA gene consisted of an initial denaturing step of 94° C. for 3 min, followed by 35 cycles of 94° C. for 45 s, 50° C. for 1 min, and 72° C. for 90 s, and a final elongation step of 72° C. for 10 min. Blank controls, in which no DNA was added to the reaction, were performed for quality assurance. Replicate amplicons were pooled and visualized by electrophoresis through 1.2% (wt/vol) agarose gels stained with 0.5 mg/mL ethidium bromide. Amplicons were purified with a PCR DNA extraction kit (IBI Scientific, Peosta, Iowa, USA) and the purified 16S rRNA amplicons were quantified using the Qubit dsDNA BR assay kit (Life Technologies, Carlsbad, Calif., USA) and a Qubit fluorometer (Life Technologies).

### Sequence Library Analysis and Statistical Analysis

Amplicon DNA aliquots were standardized to the same concentration and then pooled. Final equimolar libraries were sequenced using the MiSeq reagent kit v2 (300 cycles) on the Illumina MiSeq platform. The obtained 16S rRNA gene sequences were processed using the open source software pipeline Quantitative Insights Into Microbial Ecology (QIIME) version 1.7.0-dev [29]. Sequences were filtered for quality using established guidelines [30]. Sequences were binned into Operational Taxonomic Units (OTU) based on 97% identity using UCLUST [31] against the Greengenes reference database [32], May 2013 release. Low-abundance clusters were filtered and chimeric sequences were removed using USEARCH [31]. All samples were rarefied to an equal depth of 10,000 sequences using QIMME. The classification of reads at multiple taxonomic levels (kingdom, phylum, class, order, family, and genus) used in the present study were obtained from the MiSeq Reporter and are based on the Greengenes database cited above.

Using the obtained OTU information, each sample's richness was evaluated using the Chaol index, which is a nonparametric estimator of the minimum richness (number of OTU) and is based on the number of rare OTU (singletons and doublets) within samples. Microbiota diversity was measured using the Shannon index, which is a nonparametric diversity index that combines estimates of richness (the total number of OTU) and evenness (the relative abundance of OTU).

### β-Hydroxybutyrate Analysis

β-hydroxybutyrate was measured for 180 serum samples. The Autokit Total Ketone Bodies (Wako Pure Chemical Industries Ltd., Richmond, Va., USA), a cyclic enzymatic method based on the oxidation of BHBA to acetoacetate by BHBA dehydrogenase, was chosen to measure serum BHBA due to its high sensitivity and high specificity.

### Statistical Analysis

Pearson chi-square test was used to compare the following categorical variables between treatment groups: parity of the dam (1, 2, 3), occurrence of twins (yes or no), and calving ease of the dam (assisted or non-assisted).

Kaplan-Meier survival analysis were performed using MedCalc Statistical Software version 13.1.2 (MedCalc Software, Ostend, Belgium) to compare the effect of oral F. prausnitzii administration on the incidence of severe diarrhea cases, on the mortality rate caused by severe diarrhea and on the overall mortality rate.

The effects of oral administration of F. prausnitzii on weight gain and ADG were evaluated by linear regression models fitted in JMP with calf as the experimental unit. Variables offered to the models included treatment (control and FPTRT), birth weight, age at enrollment, age at weaning, parity of the dam (1, 2, 3), occurrence of twins, and calving ease of the dam (assisted or non-assisted). The interaction terms between treatment groups and all independent variables were evaluated in the model. Pen was fitted as a random effect. Manual backward variable elimination was undertaken considering main effects and interactions, which were retained in the model when P≤0.05.

Additionally, the relative abundance of F. prausnitzii in the 1^{st} week of life of the subset of 70 calves was dichotomized in LowFP and HighFP. The mean relative abundance of F. prausnitzii and 95% confidence intervals were 0.42% (0.30-0.54) for the LowFP calves (n=20 control, n=18 FPTRT) and 17.99% (12.99-23.00) for the HighFP calves (n=15 control, n=17 FPTRT). ANOVA was used to evaluate the effect of the low and high abundance of F. prausnitzii in the first week of life on the weight gain of this subset of calves.

Faecalibacterium, Firmicutes and Bacteroidetes mean relative abundances, Firmicutes to Bacteroidetes ratio were each compared using multiple linear mixed regression models in JMP. Variables offered to the models included treatment group, week of life, and the interaction terms between these two variables. Calf and pen were fitted as random effects. Number of OTU, Chaol and Shannon indexes means were estimated using a similar linear mixed regression model described above.

Additionally, the effect of treatment group on milk production was assessed by repeated measures ANOVA. Mixed general linear model was fitted to the data by using the mixed procedure of SAS. The outcome variable was weekly milk weights which was modeled as Gaussian (normally distributed data) variable. The assumption that the residuals were normally distributed was assessed by visually evaluating the distribution plot of the studentized residuals. Our data was longitudinally collected and therefore had a series of repeated measures of TDM throughout lactation. This implies that data points were correlated within each research subject. To account appropriately for within-cow correlation of the TDM, we modeled the error term by imposing a first-order autoregressive covariance structure for all statistical models. The model described below was fitted to the data in this study.

Y=Xβ+e

Y=weekly milk weight average

X=the matrix of all independent variables.

β=the vector of all fixed-effect parameters

e=random residual. The within-cow correlation of the TDM was accounted for by imposing a first-order autoregressive covariance structure (assuming that the within-cow correlation of the repeated measures of milk weights decreased as time between the test dates increased) to the error term.

### Results

### Safety Trial

No adverse reactions, such as increased body temperature, heart and respiratory rates, were observed after the administration of the treatments and during the following days. All 30 bull calves survived the experimental period and there was no difference in fecal consistency score, attitude, appetite or dehydration between the four treatment groups (P≥0.05). It was concluded that it was safe to administer F. prausnitzii culture to newborn calves. Although the rectal administration was a promising way of by-passing the low pH of the abomasum and the detrimental effect of bile salts, it was not an efficient practice. Most of the infused liquid was promptly excreted by the calf. Therefore, the oral route was selected for the field trial.

### Randomized Field Trial

A total of 554 Holstein heifers were enrolled in this randomized field trial, 296 in the control group and 258 in the FPTRT group. A total of 22 were twins, 12 in the control group (4.10%) and 10 in the FPTRT group (3.89%; P=0.99). Six control calves (2.05%) and seven FPTRT calves (2.72%) were born with assistance (P=0.60). The numbers of calves born from first lactation cows were 166 for control calves (56.66%) and 124 (48.25%) for FPTRT calves; from second lactation cows were 70 in the control group (23.89%) and 72 in the FPTRT (28.02%), and from third or more lactations cows were 57 in the control group (19.45%) and 61 in the FPTRT (23.74%), P=0.14).

Calves that were treated with F. prausnitzii had significantly lower incidence of severe diarrhea over the preweaning period compared to the controls, 3.1% and 6.8%, respectively (P=0.05). Mortality rate associated with severe diarrhea was also significantly lower for FPTRT calves, 1.5%, compared to control calves, 4.4% (P=0.05), and the overall mortality was numerically lower for the FPTRT group compared to the control, 3.9% and 6.1%, respectively (P=0.17).

From the 280 rectal swabs collected, DNA was successfully extracted from 264 samples. Quality-filtered reads for 16S sequences yielded a total of 16,266,816 sequences with an average coverage of 61,617 sequences per sample. The mean number of sequences per sample and the 95% confidence interval were: 62,218 (60,209-64,227) for the control group's samples and 61,033 (59,055-63,012) for the FPTRT group's samples. The effect of the interactions between the treatment groups and the week of life on the OTU. There was a significant effect of week of life on the Chao 1 (P<0.01) and Shannon (P<0.01) indexes.

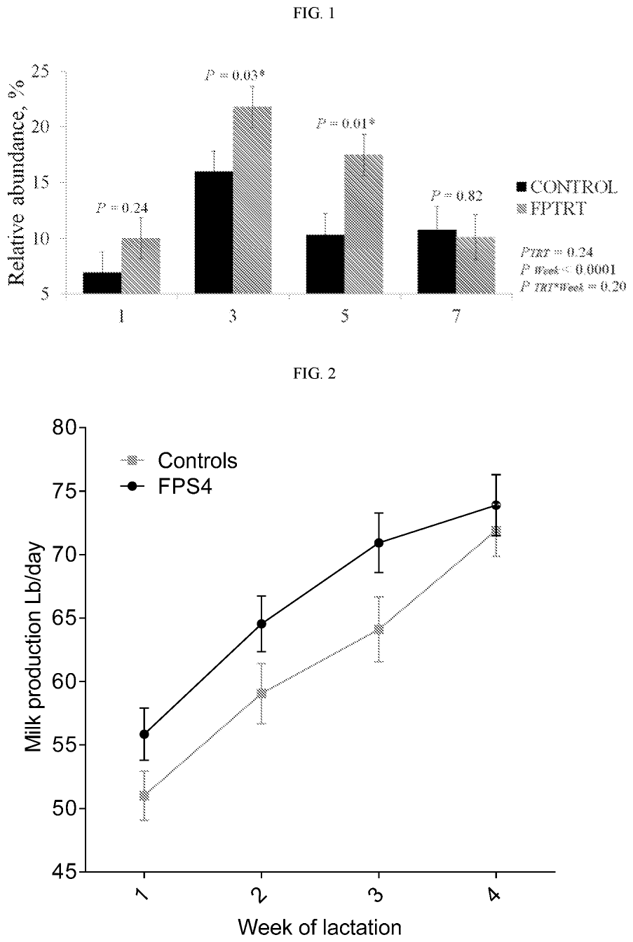

The mean relative abundance of the genus Faecalibacterium was significantly higher in the FPTRT group in the 3^{rd} and 5^{th} weeks of life (P<0.05) compared to the control group, as illustrated in FIG. 1. Other bacterial genera were not significantly different between the study groups. Faecalibacterium (mean 13.0%), Bacteroides (mean 12.2%), Ruminococcus (mean 10.8%), Blautia (mean 6.5%), and Prevotella (mean 5.6%) were the five most prevalent genera during the preweaning period. Escherichia was the 9^{th} most prevalent genus (mean 3.3%), with an average prevalence of 10% in the first week of life and decreasing to less than 0.2% in the 7^{th} week.

Calves that were treated with Faecalibacterium prausnitzii produced significantly more milk during the first 5 weeks of lactation when compared to negative control calves (FIG. 2).

### REFERENCES

1. USDA. Heifer Calf Health and Management Practices on U.S. Dairy Operations, 2007. USDA:APHIS:VS, CEAH Fort Collins, Colo. 2010: [Available online].
2. USDA. Dairy Heifer Raiser 2011. USDA. 2012: [Available online at: http://www.aphis.usda.gov].
3. World Health Organization. Antimicrobial resistance: global report on surveillance. 2014.
4. FDA. FDA introduces ‘final rule’ on use of antibiotics in feed. Vet Rec. 2015;176: 666.
5. Foditsch C, Santos T M, Teixeira A G, Pereira R V, Dias J M, Gaeta N, et al. Isolation and characterization of Faecalibacterium prausnitzii from calves and piglets. PLoS One. 2014;9: e116465.
6. Barcenilla A, Pryde S E, Martin J C, Duncan S H, Stewart C S, Henderson C, et al. Phylogenetic relationships of butyrate-producing bacteria from the human gut. Appl Environ Microbiol. 2000;66: 1654-1661.
7. Hold G L, Schwiertz A, Aminov R I, Blaut M, Flint H J. Oligonucleotide probes that detect quantitatively significant groups of butyrate-producing bacteria in human feces. Appl Environ Microbiol. 2003;69: 4320-4324.
8. Suau A, Rochet V, Sghir A, Gramet G, Brewaeys S, Sutren M, et al. Fusobacterium prausnitzii and related species represent a dominant group within the human fecal flora. Syst Appl Microbiol. 2001;24: 139-145.
9. Walker A W, Ince J, Duncan S H, Webster L M, Holtrop G, Ze X, et al. Dominant and diet-responsive groups of bacteria within the human colonic microbiota. ISME J. 2011;5: 220-230.
10. Oikonomou G, Teixeira A G, Foditsch C, Bicalho M L, Machado V S, Bicalho R C. Fecal microbial diversity in pre-weaned dairy calves as described by pyrosequencing of metagenomic 16S rDNA. Associations of Faecalibacterium species with health and growth. PLoS One. 2013;8: e63157.
11. Haenen D, Zhang J, Souza da Silva C, Bosch G, van der Meer I M, van Arkel J, et al. A diet high in resistant starch modulates microbiota composition, SCFA concentrations, and gene expression in pig intestine. J Nutr. 2013;143: 274-283.
12. Nava G M, Stappenbeck T S. Diversity of the autochthonous colonic microbiota. Gut Microbes. 2011;2: 99-104.
13. Lund M, Bjerrum L, Pedersen K. Quantification of Faecalibacterium prausnitzii—and Subdoligranulum variabile-like bacteria in the cecum of chickens by real-time PCR. Poult Sci. 2010;89: 1217-1224.
14. Balamurugan R, George G, Kabeerdoss J, Hepsiba J, Chandragunasekaran A M, Ramakrishna B S. Quantitative differences in intestinal Faecalibacterium prausnitzii in obese Indian children. Br J Nutr. 2010;103: 335-338.
15. Wang W, Chen L, Zhou R, Wang X, Song L, Huang S, et al. Increased Proportion of Bifidobacterium and the Lactobacillus group and Loss of Butyrate-producing Bacteria in Inflammatory Bowel Disease. J Clin Microbiol. 2013.
16. Sokol H, Pigneur B, Watterlot L, Lakhdari O, Bermudez-Humaran L G, Gratadoux J J, et al. Faecalibacterium prausnitzii is an anti-inflammatory commensal bacterium identified by gut microbiota analysis of Crohn disease patients. Proc Natl Acad Sci USA. 2008;105: 16731-16736.
17. Machiels K, Joossens M, Sabino J, De Preter V, Arijs I, Eeckhaut V, et al. A decrease of the butyrate-producing species Roseburia hominis and Faecalibacterium prausnitzii defines dysbiosis in patients with ulcerative colitis. Gut. 2013.
18. Qiu X, Zhang M, Yang X, Hong N, Yu C. Faecalibacterium prausnitzii upregulates regulatory T cells and anti-inflammatory cytokines in treating TNBS-induced colitis. J Crohns Colitis. 2013;7: e558-68.
19. Martin R, Miguel S, Chain F, Natividad J M, Jury J, Lu J, et al. Faecalibacterium prausnitzii prevents physiological damages in a chronic low-grade inflammation murine model. BMC Microbiol. 2015;15: 67-015-0400-1.
20. Miguel S, Leclerc M, Martin R, Chain F, Lenoir M, Raguideau S, et al. Identification of metabolic signatures linked to anti-inflammatory effects of Faecalibacterium prausnitzii. MBio. 2015;6: 10.1128/mBio.00300-15.
21. Segain J P, Raingeard de la Bletiere D, Bourreille A, Leray V, Gervois N, Rosales C, et al. Butyrate inhibits inflammatory responses through NFkappaB inhibition: implications for Crohn's disease. Gut. 2000;47: 397-403.
22. Ahmed A F, Constable P D, Misk N A. Effect of feeding frequency and route of administration on abomasal luminal pH in dairy calves fed milk replacer. J Dairy Sci. 2002;85: 1502-1508.
23. Minato H, Otsuka M, Shirasaka S, Itabashi H, and Mitsumori M. Colonization of microorganisms in the rumen of young calves. J Gen Appl Microbiol. 1992;38: 447-456.
24. Beharka A A, Nagaraja T G, Morrill J L, Kennedy G A, Klemm R D. Effects of form of the diet on anatomical, microbial, and fermentative development of the rumen of neonatal calves. J Dairy Sci. 1998;81: 1946-1955.
25. Jami E, Israel A, Kotser A, Mizrahi I. Exploring the bovine rumen bacterial community from birth to adulthood. ISME J. 2013;7: 1069-1079.
26. Keith E A, Windle L M, Keith N K, Gough R H. Feeding value of fermented waste milk with or without sodium bicarbonate for dairy calves. J Dairy Sci. 1983;66: 833-839.
27. Caporaso J G, Lauber C L, Walters W A, Berg-Lyons D, Huntley J, Fierer N, et al. Ultra-high-throughput microbial community analysis on the Illumina HiSeq and MiSeq platforms. ISME J. 2012;6: 1621-1624.
28. Gilbert J A, Meyer F, Antonopoulos D, Balaji P, Brown C T, Brown C T, et al. Meeting report: the terabase metagenomics workshop and the vision of an Earth microbiome project. Stand Genomic Sci. 2010;3: 243-248.
29. Caporaso J G, Kuczynski J, Stombaugh J, Bittinger K, Bushman F D, Costello E K, et al. QIIME allows analysis of high-throughput community sequencing data. Nat Methods. 2010;7: 335-336.
30. Bokulich N A, Subramanian S, Faith J J, Gevers D, Gordon J I, Knight R, et al. Quality-filtering vastly improves diversity estimates from Illumina amplicon sequencing. Nat Methods. 2013;10: 57-59.
31. Edgar R C. Search and clustering orders of magnitude faster than BLAST. Bioinformatics. 2010;26: 2460-2461.
32. McDonald D, Price M N, Goodrich J, Nawrocki E P, DeSantis T Z, Probst A, et al. An improved Greengenes taxonomy with explicit ranks for ecological and evolutionary analyses of bacteria and archaea. ISME J. 2012;6: 610-618.
33. Guilloteau P, Martin L, Eeckhaut V, Ducatelle R, Zabielski R, Van Immerseel F. From the gut to the peripheral tissues: the multiple effects of butyrate. Nutr Res Rev. 2010;23: 366-384.
34. Ploger S, Stumpff F, Penner G B, Schulzke J D, Gabel G, Martens H, et al. Microbial butyrate and its role for barrier function in the gastrointestinal tract. Ann N Y Acad Sci. 2012;1258: 52-59.
35. Mentschel J, Leiser R, Mulling C, Pfarrer C, Claus R. Butyric acid stimulates rumen mucosa development in the calf mainly by a reduction of apoptosis. Arch Tierernahr. 2001;55: 85-102.
36. Gorka P, Kowalski Z M, Pietrzak P, Kotunia A, Jagusiak W, Zabielski R. Is rumen development in newborn calves affected by different liquid feeds and small intestine development? J Dairy Sci. 2011;94: 3002-3013.
37. Gorka P, Kowalski Z M, Pietrzak P, Kotunia A, Jagusiak W, Holst J J, et al. Effect of method of delivery of sodium butyrate on rumen development in newborn calves. J Dairy Sci. 2011;94: 5578-5588.
38. Baldwin R L, McLeod K R, Klotz J L, Heitmann R N. Rumen Development, Intestinal Growth and Hepatic Metabolism In The Pre- and Postweaning Ruminant. J Dairy Sci. 2004;87:(E. Suppl.): E55-E65.
39. Bischoff S C, Barbara G, Buurman W, Ockhuizen T, Schulzke J D, Serino M, et al. Intestinal permeability-a new target for disease prevention and therapy. BMC Gastroenterol. 2014;14: 189-014-0189-7.
40. Hollander D. Intestinal permeability, leaky gut, and intestinal disorders. Curr Gastroenterol Rep. 1999;1: 410-416.
41. Geurts L, Neyrinck A M, Delzenne N M, Knauf C, Cani P D. Gut microbiota controls adipose tissue expansion, gut barrier and glucose metabolism: novel insights into molecular targets and interventions using prebiotics. Benef Microbes. 2014;5: 3-17.
42. Turnbaugh P J, Ley R E, Mahowald M A, Magrini V, Mardis E R, Gordon J I. An obesity-associated gut microbiome with increased capacity for energy harvest. Nature. 2006;444: 1027-1031.
43. Ley R E, Backhed F, Turnbaugh P, Lozupone C A, Knight R D, Gordon J I. Obesity alters gut microbial ecology. Proc Natl Acad Sci USA. 2005;102: 11070-11075.
44. Soberon F, Raffrenato E, Everett R W, Van Amburgh M E. Preweaning milk replacer intake and effects on long-term productivity of dairy calves. J Dairy Sci. 2012;95: 783-793.
45. Soberon F, Van Amburgh M E. Lactation Biology Symposium: The effect of nutrient intake from milk or milk replacer of preweaned dairy calves on lactation milk yield as adults: a meta-analysis of current data. J Anim Sci. 2013;91: 706-712.
46. Hill T M, Bateman H G,2nd, Aldrich J M, Schlotterbeck R L. Effect of milk replacer program on digestion of nutrients in dairy calves. J Dairy Sci. 2010;93: 1105-1115.
47. Bruss M L. Lipids and Ketones. Clinical Biochemistry of Domestic Animals (5th ed). 1997: 83-115.
48. Steele M A, Vandervoort G, AlZahal O, Hook S E, Matthews J C, McBride B W. Rumen epithelial adaptation to high-grain diets involves the coordinated regulation of genes involved in cholesterol homeostasis. Physiol Genomics. 2011;43: 308-316.
49. Malmuthuge N, Griebel P J, Guan le L. Taxonomic identification of commensal bacteria associated with the mucosa and digesta throughout the gastrointestinal tracts of preweaned calves. Appl Environ Microbiol. 2014;80: 2021-2028.
50. Galvao K N, Santos J E, Coscioni A, Villasenor M, Sischo W M, Berge A C. Effect of feeding live yeast products to calves with failure of passive transfer on performance and patterns of antibiotic resistance in fecal Escherichia coli. Reprod Nutr Dev. 2005;45: 427-440.
51. Ślusarczyk K, Strzetelski J A, Furgal-Dierźuk I. The effect of sodium butyrate on calf growth and serum level of β-hydroxybutyric acid. Journal of Animal and Feed Sciences. 2010;19: 348-357.
52. Klein-Jobstl D, Schornsteiner E, Mann E, Wagner M, Drillich M, Schmitz-Esser S. Pyrosequencing reveals diverse fecal microbiota in Simmental calves during early development. Front Microbiol. 2014;5: 622.
53. Edrington T S, Dowd S E, Farrow R F, Hagevoort G R, Callaway T R, Anderson R C, et al. Development of colonic microflora as assessed by pyrosequencing in dairy calves fed waste milk. J Dairy Sci. 2012;95: 4519-4525.

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

1. A method of increasing milk production in an animal comprising administering Faecalibacterium spp. to the animal under conditions such that milk production in the animal is increased.

2. The method of claim 1, wherein said Faecalibacterium spp. is Faecalibacterium prausnitzii.

3. The method of claim 1, wherein said composition comprises one or more Faecalibacterium spp. in an amount effective to improve milk production in said animal.

4. The method of claim 1, wherein said composition comprises one or more Faecalibacterium spp. in an amount effective increase future milk production in said animal.

5. The method of claim 1, wherein said animal is a domestic animal.

6. The method of claim 5, wherein said domestic animal is selected from the group consisting of cattle, sheep, swine, goats, and horses.

7. The method of claim 1, wherein said animal is a calf.

8. The method of claim 1, wherein said animal is less than 1 week of age.

9. The method of claim 1, wherein said animal is less than one month of age.

10. The method of claim 1, wherein said animal is less than two months of age.

11. The method of claim 1, wherein said composition is formulated as a powder, bolus, gel, drench, or capsule.

12. The method of claim 1, wherein said composition is provided as part of a milk replacer.

13. The method of claim 1, wherein said composition is coadministered with at least a second probiotic organism selected from the group consisting of Lactobacillus acidophilus, L. lactis, L. plantarum, L. casei, Bacillus subtilis, B. lichenformis, Enterococcus faecium, Bifidobacterium bifidum, B. longum, B. thermophilum, Propionibacterium jensenii, yeast, and combinations thereof.

14. The method of claim 1, wherein said composition is formulated with an additional additive selected from the group consisting of an energy substrate, a mineral, a vitamin, and combinations thereof.
